(19) United States
(12) Patent Application Publication
(10) Pub. No.: US 2025/0387052 A1
(43) Pub. Date: Dec. 25, 2025

(54) IMAGE PROCESSING APPARATUS, ENDOSCOPE SYSTEM, AND OPERATION METHOD PERFORMED BY OPERATING IMAGE PROCESSING APPARATUS

(71) Applicants: OLYMPUS MEDICAL SYSTEMS CORP., Tokyo (JP); NATIONAL CANCER CENTER, Tokyo (JP)

(72) Inventors: Tomoko ITO, Tokyo (JP); Keigo MATSUO, Tokyo (JP); Kazunari HANANO, Tokyo (JP); Tetsuhide TAKEYAMA, Tokyo (JP); Taku SAKAMOTO, Tokyo (JP); Naoya TOYOSHIMA, Tokyo (JP); Ichiro ODA, Tokyo (JP)

(73) Assignee: OLYMPUS MEDICAL SYSTEMS CORP., Tokyo (JP)

(21) Appl. No.: 19/181,678

(22) Filed: Apr. 17, 2025

(30) Foreign Application Priority Data
Jun. 24, 2024 (JP) 2024-101397

(51) Int. Cl. A61B 5/1455; A61B 1/00

(52) U.S. Cl. CPC A61B 5/14552; A61B 1/000094

(57) ABSTRACT

An image processing apparatus includes: a processor including hardware, the processor being configured to acquire a first observation image obtained by capturing an image of return light of first observation light applied to a subject; and estimate a depth of invasion based on a pixel value of each pixel of the first observation image.

### CROSS-REFERENCE TO RELATED APPLICATION(S)

This application is based upon and claims the benefit of priority from Japanese Patent Application No. 2024-101397, filed on Jun. 24, 2024, the entire contents of which are incorporated herein by reference.

### BACKGROUND

### 1. Technical Field

The present disclosure relates to an image processing apparatus, an endoscope system, and an operation method performed by an image processing apparatus.

### 2. Related Art

There has been the case where red light is applied to a subject in endoscopic observation (for example, refer to Japanese Laid-open Patent Publication No. 2022-036326). There has also been the case where an endoscopist, such as a doctor, determines a depth of invasion using endoscopic observation.

### SUMMARY

In some embodiments, an image processing apparatus includes: a processor including hardware, the processor being configured to acquire a first observation image obtained by capturing an image of return light of first observation light applied to a subject; and estimate a depth of invasion based on a pixel value of each pixel of the first observation image.

In some embodiments, an endoscope system includes: a light source configured to apply first observation light to a subject from a distal end of an insertion portion to be inserted into the subject; an imager configured to generate a first observation image obtained by capturing an image of return light of the first observation light; and an image processing apparatus including a processor including hardware, the processor being configured to acquire the first observation image from the imager and estimate a depth of invasion based on a pixel value of each pixel of the first observation image.

In some embodiments, provided is an operation method performed by an image processing apparatus. The method includes: acquiring a first image obtained by capturing an image of return light of first observation light applied to a subject; and estimating a depth of invasion based on a pixel value of each pixel of the first observation image.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an entire configuration of an endoscope system including an image processing apparatus according to Embodiment 1-1;

FIG. 2 is a block diagram illustrating a functional configuration of a relevant part of an endoscope and a control device according to Embodiment 1-1;

FIG. 3 is a chart illustrating the spectral reflectance of each type of tissue;

FIG. 4 is a flowchart illustrating an overview of a process that the image processing apparatus executes according to Embodiment 1-1;

FIG. 5 is a chart illustrating an absorption spectrum of hemoglobin;

FIG. 6 is a chart illustrating that cancerous and non-cancerous spectral reflectance is approximated by a linear expression;

FIG. 7 is a diagram illustrating an example of a depth-of-invasion map;

FIG. 8 is a diagram illustrating a modification of the depth-of-invasion map;

FIG. 9 is a flowchart illustrating an overview of a process that an image processing apparatus executes according to Embodiment 1-2;

FIG. 10 is a chart illustrating that cancerous and non-cancerous spectral reflectance is approximated by two linear expressions;

FIG. 11 is a chart illustrating that cancerous and non-cancerous spectral reflectance is approximated by a linear expression;

FIG. 12 is a block diagram illustrating a functional configuration of a relevant part of the endoscope and a control device according to Embodiment 2-1;

FIG. 13 is a flowchart illustrating an overview of a process that the image processing apparatus executes according to Embodiment 2-1;

FIG. 14 is a diagram illustrating a difference in pixel value between a R signal and an Am signal;

FIG. 15 is a diagram illustrating that the pixel values of the R signal and the Am signal are corrected;

FIG. 16 is a flowchart illustrating an overview of a process that an image processing apparatus executes according to Embodiment 2-2;

FIG. 17 is a flowchart illustrating an overview of a process that an image processing apparatus executes according to Embodiment 2-3;

FIG. 18 is a flowchart illustrating an overview of a process that an image processing apparatus executes according to Embodiment 2-4;

FIG. 19 is a block diagram illustrating a functional configuration of a relevant part of an endoscope and a control device according to Embodiment 3-1;

FIG. 20 is a flowchart illustrating an overview of a process that an image processing apparatus executes according to Embodiment 3-1;

FIG. 21 is a flowchart illustrating an overview of a process that an image processing apparatus executes according to Embodiment 3-2;

FIG. 22 is a block diagram illustrating a functional configuration of a relevant part of the endoscope and a control device according to Embodiment 4-1;

FIG. 23 is a flowchart illustrating an overview of a process that an image processing apparatus executes according to Embodiment 4-1;

FIG. 24 is a flowchart illustrating an overview of a process that an image processing apparatus executes according to Embodiment 4-2;

FIG. 25 is a flowchart illustrating an overview of a process that an image processing apparatus executes according to Embodiment 4-3;

FIG. 26 is a flowchart illustrating an overview of a process that an image processing apparatus executes according to Embodiment 4-4;

FIG. 27 is a flowchart illustrating an overview of a process that an image processing apparatus executes according to Embodiment 5-1;

FIG. 28 is a flowchart illustrating an overview of a process that an image processing apparatus executes according to Embodiment 5-2;

FIG. 29 is a flowchart illustrating an overview of a process that an image processing apparatus executes according to Embodiment 6-1;

FIG. 30 is a flowchart illustrating an overview of a process that an image processing apparatus executes according to Embodiment 6-2;

FIG. 31 is a flowchart illustrating an overview of a process that an image processing apparatus executes according to Embodiment 6-3; and

FIG. 32 is a flowchart illustrating an overview of a process that an image processing apparatus executes according to Embodiment 6-4.

### DETAILED DESCRIPTION

Embodiments of an image processing apparatus, an endoscope system, and an operation method performed by an image processing apparatus according to the present disclosure will be described below with reference to the accompanying drawings. Note that the embodiments do not limit the disclosure. The present disclosure is generally applicable to an image processing apparatus, an endoscope system, and an operation method performed by an image processing apparatus that are used to determine a depth of invasion.

In the illustration of the drawings, the same elements are denoted with the same reference numerals appropriately. It is necessary to note that the drawings are schematic and the relation of each element in size, the proportion of each element, etc., are sometimes different from actual ones. Parts of which relation in size and of which ratio differs between drawings may be contained in the drawings.

### EMBODIMENT 1-1

### Configuration of Endoscope System

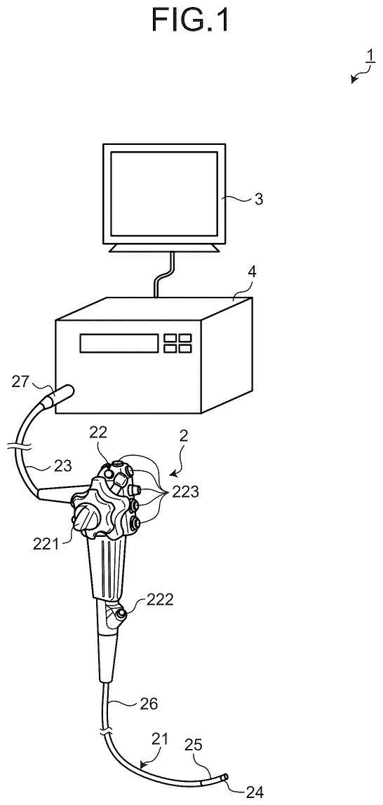

FIG. 1 is a diagram illustrating an entire configuration of an endoscope system including an image processing apparatus according to Embodiment 1-1. An endoscope system 1, for example, is a system that is used in the medical fields and that observers the inside of a subject (a living body). The endoscope system 1 includes an endoscope 2, a display 3, and a control device 4.

The endoscope 2 sequentially generates image data (RAW data) by capturing internal images of the subject and sequentially outputs the image data to the control device 4. As illustrated in FIG. 1, the endoscope 2 includes an insertion portion 21, an operation portion 22, and a universal cord 23.

The insertion portion 21 is at least partly flexible and that is inserted into the subject. As illustrated in FIG. 1, the insertion portion 21 includes a distal end part 24 that is arranged at a distal end of the insertion portion 21, a bendable portion 25 that is connected to a proximal end side of the distal end unit 24 (the side of the operation portion 22) and that is configured to be bendable, and a flexible tube 26 that is flexible and elongated and that is connected to a proximal end side of the bendable portion 25.

The operation portion 22 is connected to a proximal end portion of the insertion portion 21. The operation portion 22 receives various types of operations on the endoscope 2. As illustrated in FIG. 1, the operation portion 22 is provided with a bending knob 221, an insertion port 222, and a plurality of operation parts 223.

The bending knob 221 is configured to be rotatable according to a user operation performed by a user, such as an endoscopist. A rotation of the bending knob 221 causes a bendable mechanism (not illustrated in the drawings), such as a wire that is arranged in the insertion portion 21 and that is made of metal or resin, to operate. Accordingly, the bendable portion 25 bends.

The insertion port 222 and a treatment tool channel (not illustrated in the drawings) that is a conduit extending from the distal end of the insertion portion 21 communicate and the insertion port 222 is an insertion port for inserting a treatment tool, or the like, into the treatment tool channel from the outside of the endoscope 2.

The operation parts 223 consist of buttons that receive various types of operations performed by the user, such as an endoscopist, and outputs operation signals corresponding to the various types of operations to the control device 4 via the universal cord 23. A release operation of making an instruction to capture a still image with the endoscope 2 and an operation of switching an observation mode of the endoscope 2 between a normal light observation mode and a special observation mode can be exemplified as the various types of operations.

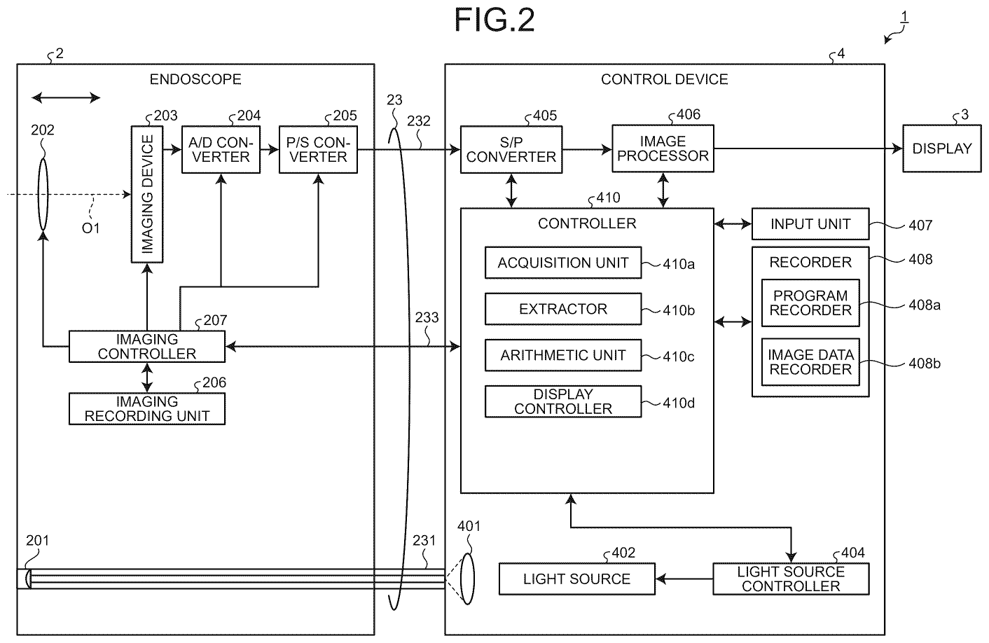

The universal cord 23 is a cord that extends from the operation portion 22 in a direction different from the direction in which the insertion portion 21 extends and, in the universal cord 23, a light guide 231 (refer to FIG. 2) consisting of optical fibers, or the like, a first signal line 232 (refer to FIG. 2) that transmits the above-described image data, and a second signal line 233 (refer to FIG. 2) that transmits the above-described operation signals, and the like, etc., are arranged. As illustrated in FIG. 1, a connector 27 is arranged at a proximal end of the universal cord 23. The connector 27 is detachably connected to the control device 4.

The display 3 consists of a display monitor of liquid crystals, organic electro luminescence (EL), or the like, and, under the control of the control device 4, displays a display image based on the image data on which image processing has been performed in the control device 4 and various types of information on the endoscope 2.

The control device 4 is realized using a processor that is a processing unit including hardware, such as a graphics processing unit (GPU), a field programmable gate array (FPGA), or a central processing unit (CPU), and a memory that is a temporal storage that the processor uses. The control device 4 generally controls operations of each unit of the endoscope 2 according to a program that is recorded in the memory.

### Functional Configuration of Relevant Part of Endoscope System

A functional configuration of a relevant part of the above-described endoscope system 1 will be described next. FIG. 2 is a block diagram illustrating a functional configuration of a relevant part of an endoscope and a control device according to Embodiment 1-1. The endoscope 2 and the control device 4 will be described below in sequence.

### Functional Configuration of Endoscope

First of all, a functional configuration of the endoscope 2 will be described. As illustrated in FIG. 2, the endoscope 2 includes an illumination optical system 201, an imaging optical system 202, an imaging device 203 serving as an imager, a A/D converter 204, a P/S converter 205, an imaging recording unit 206, and an imaging controller 207. Each of the illumination optical system 201, the imaging optical system 202, the imaging device 203, the A/D converter 204, the P/S converter 205, the imaging recording unit 206, and the imaging controller 207 is arranged in the distal end part 24.

The illumination optical system 201 consists of at least one lens and applies illuminating light that is supplied from the light guide 231 to an object.

The imaging optical system 202 is configured using a plurality of lenses and an actuator consisting of a stepping motor or a voice coil motor that causes a predetermined one of the lenses to move in an optical direction. The imaging optical system 202 collects light, such as reflected light that is reflected from the object, return light from the object, and light, such as fluorescence, that the object emits and thereby forms an object image on a light receiving surface of the imaging device 203.

The imaging device 203 is configured using an image sensor, such as a charge coupled device (CCD) obtained by arranging any one of color filters forming the Bayer arrangement (RGGB) in each of a plurality of pixels that are arranged in a two-dimensional matrix form or complementary metal oxide semiconductor (CMOS). Under the control of the imaging controller 207, the imaging device 203 receives light of the object image that is formed by the imaging optical system 202 and performs photoelectric conversion, thereby generating a captured image (analog signal). The imaging device 203 outputs image data to the A/D converter 204.

The A/D converter 204 is configured using an A/D converter circuit, or the like. Under the control of the imaging controller 207, the A/D converter 204 performs A/D conversion processing on the analog image data that is input from the imaging device 203 and outputs the processed image data to the P/S converter 205.

The P/S converter 205 is configured using a P/S conversion circuit, or the like. Under the control of the imaging controller 207, the P/S converter 205 performs parallel/serial conversion on the digital image data that is input from the A/D converter 204 and outputs the image data to the control device 4 via the first signal line 232. Note that a configuration in which an E/O converter that converts image data into an optical signal is provided instead of the P/S converter 205 and the image data is output to the control device 4 using the optical signal may be employed. For example, a configuration in which image data is transmitted to the control device 4 by wireless communication, such as Wi-Fi (Wireless Fidelity)) (trademark) may be employed.

The imaging recording unit 206 is configured using a non-volatile memory or a volatile memory and records various types of information on the endoscope 2 (for example, pixel information on the imaging device 203). The imaging recording unit 206 records various types of setting data and parameters for control that are transmitted from the control device 4 via the second signal line 233.

The imaging controller 207 is realized using a timing generator (TG), a processor that is a processing device including hardware, such as a CPU, and a memory that is a temporary storage area that the processor uses. The imaging controller 207 controls operations of each of the imaging device 203, the A/D converter 204, and the P/S converter 205 based on the setting data that is received from the control device 4 via the second signal line 233.

### Functional Configuration of Control Device

A functional configuration of the control device 4 will be described next. As illustrated in FIG. 2, the control device 4 includes a condenser lens 401, a light source 402, a light source controller 404, a S/P converter 405, an image processor 406, an input unit 407, a recorder 408, and a controller 410.

The condenser lens 401 converges light that is emitted by the light source 402 and emits the converged light to the light guide 231.

Under the control of the light source controller 404, the light source 402 emits white light (normal light) that is visible light, thereby supplying the white light as the illuminating light to the light guide 231. The light source 402 is configured using a white light emitting diode (LED) lamp, a driver, etc. The light source 402 may simultaneously emit light with a red LED lamp, a green LED lamp, and a blue LED lamp, thereby supplying white light that is visible light. The light source 402 may be configured using a halogen lamp, a xenon lamp, or the like.

The light source controller 404 is realized using a processor including hardware, such as a FPGA or a CPU, and a memory that is a temporary storage that the processor uses. Based on control data that is input from the controller 410, the light source controller 404 controls light emission timing, the time of light emission, etc.

Under the control of the controller 410, the S/P converter 405 performs serial/parallel conversion on the image data that is received from the endoscope 2 via the first signal line 232 and outputs the converted image data to the image processor 406. Note that, in the case where the endoscope 2 outputs the image data in an optical signal, an O/E converter that converts optical signal into an electric signal may be provided instead of the S/P converter 405. In the case where the endoscope 2 transmits the image data by wireless communication, a communication module capable of receiving a radio signal may be provided instead of the S/P converter 405.

The image processor 406 is realized using a processor including hardware, such as a GPU or a FPGA, and a memory that is a temporary storage that the processor uses. Under the control of the controller 410, the image processor 406 performs predetermined image processing on the image data of parallel data that is input from the S/P converter 405 and outputs the processed image data to the display 3. Demosaic processing, white balance processing, gain adjustment processing, γ correction processing, format conversion processing, etc., can be exemplified as the predetermined image processing.

The input unit 407 is configured using a mouse, a foot switch, a keyboard, a button, a switch, a touch panel, etc. The input unit 407 receives a user operation performed by the user, such as an endoscopist, and outputs the received operation to the controller 410.

The recorder 408 is configured using a volatile memory, a non-volatile memory, a solid state drive (SSD), and a hard disk drive (HDD) or a recording medium, such as a memory card. The recorder 408 records data containing various types of parameters necessary for operations of the control device 4 and the endoscope 2. The recorder 408 includes a program recorder 408a that records various types of programs for running the endoscope 2 and the control device 4 and an image data recorder 408b that records an image file that stores an image corresponding to image data.

Under the control of the controller 410, the image data recorder 408b records a group of sets of image data generated by the endoscope by capturing images of a plurality of sites of observation of the subject in association with patient information, or the like.

The controller 410 corresponds to the image processing apparatus according to the disclosure. The controller 410 is realized using a processor including hardware, such as a FPGA or a CPU, and a memory that is a temporary storage that the processor uses. The controller 410 generally controls each of the units forming the endoscope 2 and the control device 4. The controller 410 includes an acquisition unit 410a, an extractor 410b, an arithmetic unit 410c, and a display controller 410d.

The acquisition unit 410a acquires a first observation image obtained by capturing an image of return light of first observation light applied to the subject. In Embodiment 1-1, the first observation light is white light that is emitted by the light source 402 and is a wideband light with a wideband wavelength component. The first observation image is a spectroscopic image of each wavelength.

The extractor 410b extracts a pixel value of each pixel of the spectroscopic image in a predetermined wavelength band. The predetermined wavelength band, for example, is from 600 nm to 700 nm, and it may be a wavelength band contained in the wavelengths from 600 nm to 700 nm. The lower limit of the predetermined wavelength band is preferably a wavelength where a difference in change between an absorption coefficient of deoxygenated hemoglobin and an absorption coefficient of oxygenated hemoglobin at each wavelength occurs.

The arithmetic unit 410c estimates a depth of invasion based on the pixel value in each pixel of the first observation image. Specifically, the arithmetic unit 410c estimates a depth of invasion based on a slope of a spectroscopic spectrum that is generated from the pixel values that are extracted by the extractor 410b.

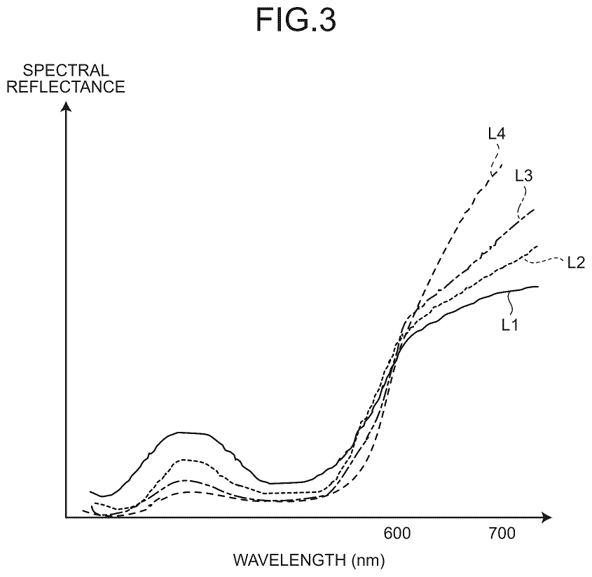

FIG. 3 is a chart illustrating the spectral reflectance of each type of tissue. The vertical axis in FIG. 3 represents the wavelength and the horizontal axis represents the spectral reflectance of each type of tissue. The lines L1 to L4 in FIG. 3 represent the spectral reflectances of sets of tissue where depths of invasion are non-cancerous, M, SM, and MP, respectively. The depths of invasion are in the descending order of MP>SM>M>non-cancerous and the slope of the spectroscopic spectrum is large in this order. Using such characteristics, the arithmetic unit 410c estimates a depth of invasion.

The display controller 410d causes the display 3 to display a depth of invasion of each pixel. The display controller 410d causes the display 3 to display the depth of invasion of each pixel in a superimposed manner on the first observation image or a normal light image.

### Process Performed by Image Processing Apparatus

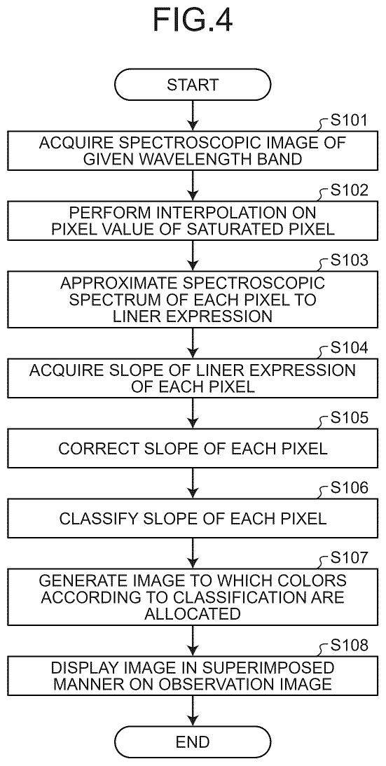

An overview of a process that the image processing apparatus executes will be described next. FIG. 4 is a flowchart illustrating the overview of the process that the image processing apparatus executes according to Embodiment 1-1.

As illustrated in FIG. 4, first of all, the acquisition unit 410a acquires a spectroscopic image of the predetermined wavelength band (from the wavelength of 600 nm to the wavelength of 700 nm) that is extracted by the extractor 410b (step S101).

Subsequently, the arithmetic unit 410c performs interpolation on a pixel value of a saturated pixel using the pixel values of the surrounding pixels (step S102). The saturated pixel is a pixel of which signal level is saturated. This pre-processing reduces variations among the pixels.

Thereafter, the arithmetic unit 410c approximates

the spectroscopic spectrum of each pixel to a linear expression (step S103). The arithmetic unit 410c sets the slope of the linear expression for an index of estimation of a depth of invasion.

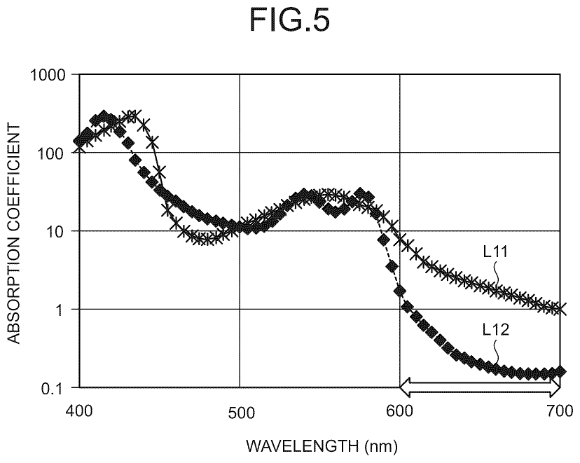

FIG. 5 is a chart illustrating an absorption

spectrum of hemoglobin. The horizontal axis in FIG. 5 represents the wavelength and the vertical axis represents the absorption coefficient. The line L11 in FIG. 5 represents the absorption coefficient of deoxygenated hemoglobin and the line L12 represents the absorption coefficient of oxygenated hemoglobin. As illustrated in FIG. 5, a difference in change between the absorption coefficients of deoxygenated hemoglobin and oxygenated hemoglobin arises from a wavelength that is around the wavelength of 580 nm and where the line L11 and the line L12 intersect and the difference in change is significant between the wavelength of 600 nm and the wavelength of 700 nm where an effect of a change in the volume of blood due to canceration tends to appear.

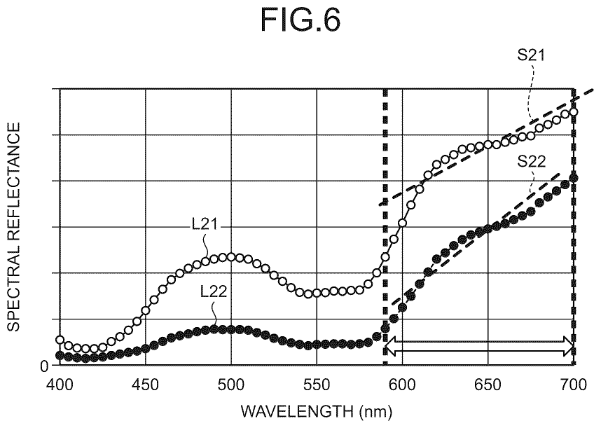

FIG. 6 is a chart illustrating that cancerous and non-cancerous spectral reflectance is approximated by a linear expression. The horizontal axis in FIG. 6 represents the wavelength and the vertical axis represents the spectral reflectance. The line L21 in FIG. 6 represents the spectral reflectance of non-cancerous tissue and the line L22 represents the spectral reflectance of cancerous tissue. Between the wavelength of 600 nm and the wavelength of 700 nm, when the slope of the straight line S21 obtained by approximating the line L21 with a liner expression and the slope of the straight line S22 obtained by approximating the line L22 by the liner expression are compared to each other, the slope of the straight line S22 is greater than the slope of the straight line S21. By determining, from the relationship, a correspondence relationship of the slopes of the liner expressions and the depths of invasion previously and storing the correspondence relationship in the recorder 408, the arithmetic unit 410c is able to estimate a depth of invasion using the slope of a liner expression. FIG. 6 proves that the slope of the straight line S22 begins to be greater than the slope of the straight line S21 from the wavelength where the line L11 and the line L12 illustrated in FIG. 5 intersect.

Back to FIG. 4, the arithmetic unit 410c corrects the calculated slope of each pixel (step S105). This post-processing corrects the slope serving as the index of estimation of a depth of invasion.

Furthermore, the arithmetic unit 410c classifies the slope of each pixel (step S106). The arithmetic unit 410c, for example, classifies the slope of each pixel into SM1 or shallower or SM2 or deeper.

The display controller 410d generates an image to which colors according to the classification are allocated (step S107). Note that the display controller 410d may generate an image that is color-coded using a value obtained by averaging a calculated slope using a plurality of pixels.

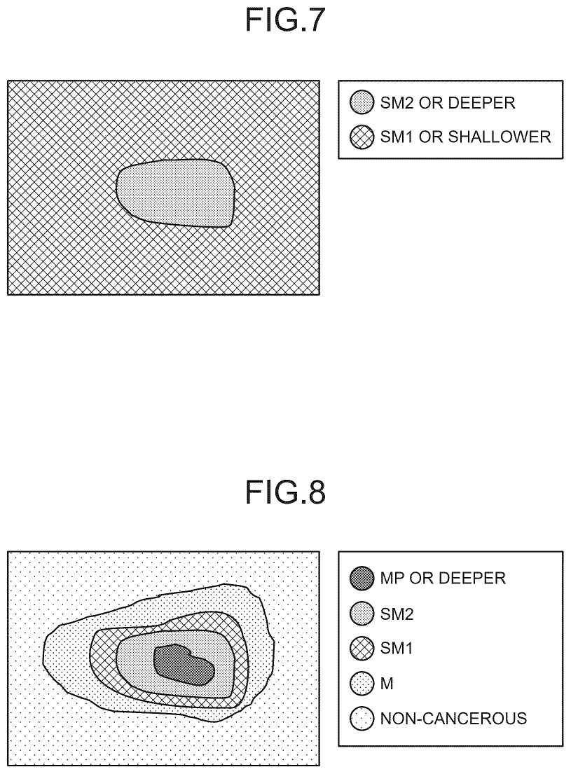

Furthermore, the display controller 410d displays the image on the observation image (normal light image) in a superimposed manner (step S108). FIG. 7 is a diagram illustrating an example of a depth-of-invasion map. As illustrated in FIG. 7, causing the display 3 to display the image that is classified into SM1 or shallower or SM2 or deeper makes it possible to assist the endoscopist in determining of a depth of invasion and increase the speed of determining a depth of invasion. The display controller 410d may superimpose only an area of the image that is color-coded according to the depths of invasion, which is an area specified by the user. Moreover, the display controller 410d may superimpose only an area of the image that is color-coded according to the depths of invasion, which is an area where a depth of invasion which is determined automatically according to a predetermined program is at or above a threshold. Furthermore, the display controller 410d may have a function of displaying a depth of invasion at a position that is specified by a cursor on the image that is color-coded according to the depths of invasion. Furthermore, the display controller 410d may have a function of displaying an average of depths of invasion in an area that is specified by the user in the image color-coded according to the depths of invasion.

According to Embodiment 1-1 described above, because the depths of cancerous invasion are estimated based on the pixel values, it is possible to assist the endoscopist in determining a depth of invasion and increase the speed of determining a depth of invasion.

FIG. 8 is a diagram illustrating a modification of the depth-of-invasion map. As illustrated in FIG. 8, depths of cancerous invasion may be estimated at five stages of MP or deeper, SM2, SM1, and Non-cancerous. As described above, depths of cancerous invasion may be classified into a plurality of stages. Not limited to the classification of SM1 or shallower or SM2 or deeper illustrated in FIG. 7, a depth of invasion may be classified into Cancerous or Non-cancerous. An image in which the color changes sequentially according to the depth of invasion (result of an arithmetic operation of a slope of liner expression) may be generated.

The display controller 410d may cause the display 3 to display various images that make it easy to see the depths of cancerous invasion. For example, the display controller 410d may make a highlighted display of an area where invasion is deep. The display controller 410d may make a highlighted display of an area where cancerous invasion is at or above a threshold. The display controller 410d may make a highlighted display of an area where it is estimated that cancer has invaded a muscular layer. The display controller 410d may make a highlighted display of an elevated area and a depressed area. The display controller 410d may generate an image obtained by combining the exemplified modes of display and cause the display 3 to display the image.

### EMBODIMENT 1-2

A process that the image processing apparatus executes according to Embodiment 1-2 will be described next. Description of the same process as the process already described will be omitted as appropriate.

The extractor 410b extracts a pixel value of each pixel of a spectroscopic image in each of two wavelength bands. The two wavelength bands are, for example, a first wavelength band between the wavelengths of 600 nm and 650 nm and a second wavelength band between the wavelengths of 650 nm and 700 nm, and the two wavelength bands may be contained in the wavelengths from 600 nm to 700 nm. The lower limits of the two wavelength bands are preferably wavelengths where a difference in change between an absorption coefficient of deoxygenated hemoglobin and an absorption coefficient of oxygenated hemoglobin at each wavelength occurs.

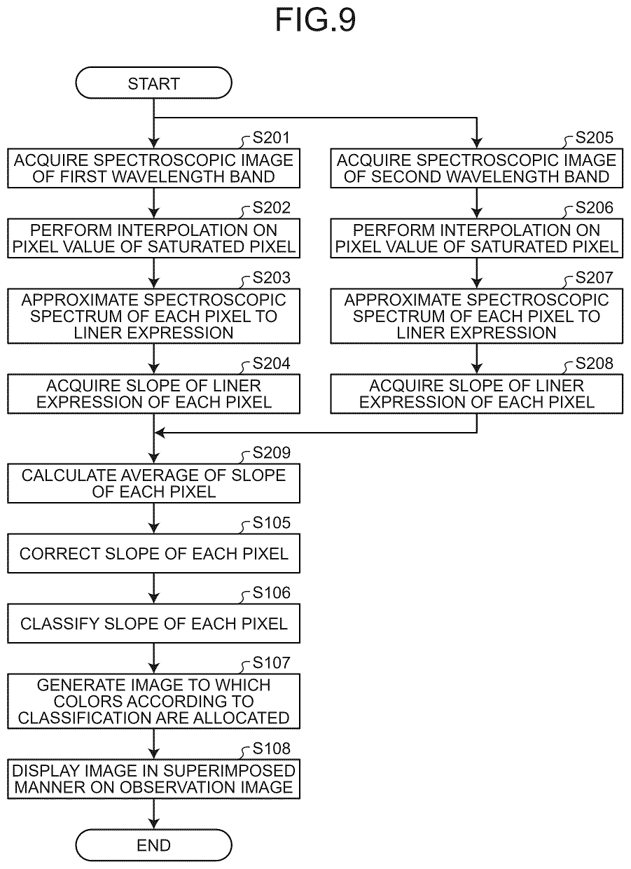

FIG. 9 is a flowchart illustrating an overview of the process that the image processing apparatus executes according to Embodiment 1-2.

As illustrated in FIG. 9, first of all, the acquisition unit 410a acquires a spectroscopic image of the first wavelength band (step S201). The first wavelength band is, for example, between the wavelengths of 600 nm and 700 nm.

Subsequently, as at step S102, the arithmetic unit 410c performs interpolation on a pixel value of a saturated pixel (step S202).

Thereafter, as at step S103, the arithmetic unit 410c approximates the spectroscopic spectrum of each pixel to a linear expression (step S203).

As at step S104, the arithmetic unit 410c acquires a slope of the linear expression of each pixel (step S204).

In line with steps S201 to S204, the process of steps S205 to S208 is executed. First of all, the acquisition unit 410a acquires a spectroscopic image of the second wavelength band (step S205). The second wavelength band is, for example, between the wavelengths of 650 nm and 700 nm.

The process of steps S206 to S208 is the same as the process of steps S202 to S204.

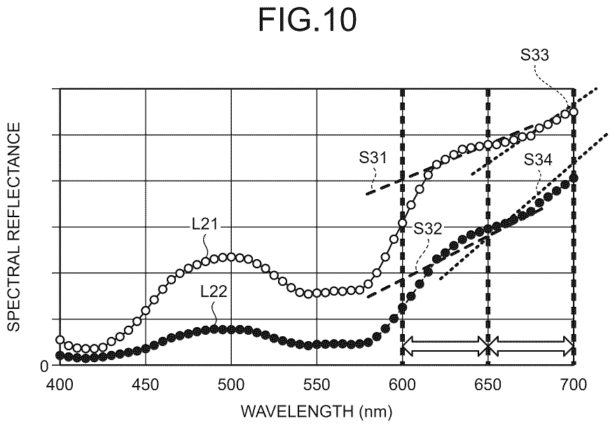

FIG. 10 is a chart illustrating that cancerous and non-cancerous spectral reflectance is approximated by two linear expressions. The horizontal axis in FIG. 10 represents the wavelength and the vertical axis represents the spectral reflectance. The line L21 in FIG. 10 represents the spectral reflectance of non-cancerous tissue and the line L22 represents the spectral reflectance of cancerous tissue. Between the wavelength of 600 nm and the wavelength of 650 nm, the arithmetic unit 410c calculates a slope of a straight line S31 obtained by approximating the line L21 by a liner expression and a slope of a straight line S32 obtained by approximating the line L22 by the liner expression. Furthermore, between the wavelength of 650 nm and the wavelength of 700 nm, the arithmetic unit 410c calculates a slope of a straight line S33 obtained by approximating the line L21 by a liner expression and a slope of a straight line S34 obtained by approximating the line L22 by the liner expression.

Subsequently, the arithmetic unit 410c calculates an average of the slope of each pixel (step S209). Dividing the wavelength band, making an approximation, and calculating an average as described above make it possible to calculate slopes appropriately.

Thereafter, as in Embodiment 1-1, the process of steps S105 to S108 is executed.

According to Embodiment 1-2 described above, because an index of estimation of a depth of invasion is calculated in divided two wavelength bands, it is possible to further increase the speed of determining a depth of invasion.

### EMBODIMENT 1-3

A process that the image processing apparatus executes according to Embodiment 1-3 will be described next. The process executed by the image processing apparatus according to Embodiment 1-3 may be the same as the process of Embodiment 1-1 illustrated in FIG. 4 and thus description thereof will be omitted. Note that, at step S101, the acquisition unit 410a acquires a spectroscopic image of a predetermined wavelength band (from the wavelength of 640 nm to the wavelength of 650 nm) that is extracted by the extractor 410b (step S101).

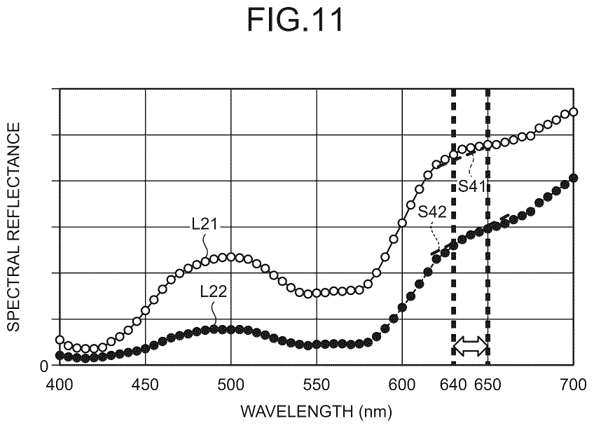

FIG. 11 is a chart illustrating that cancerous and non-cancerous spectral reflectance is approximated by a linear expression. The horizontal axis in FIG. 11 represents the wavelength and the vertical axis represents the spectral reflectance. The line L21 in FIG. 11 represents the spectral reflectance of non-cancerous tissue and the line L22 represents the spectral reflectance of cancerous tissue. As for the wavelength band between 640 nm and 650 nm, the arithmetic unit 410c calculates a slope of a straight line S41 obtained by approximating the line L21 by a liner expression and a slope of a straight line S42 obtained by approximating the line L22 by the liner expression.

Using a statistical method (T method), it was estimated that the slope of the spectroscopic spectrum and the depth of invasion have a high correlation at the wavelengths 645 nm, 650 nm, 655 nm, and 660 nm. Furthermore, the relation between the slope of the spectroscopic spectrum and the depth of invasion was checked using data on each case and it was presented that there were the fewest exceptions at 645 nm. For this reason, calculating a slope of the spectroscopic spectrum at the wavelength of 645 nm using the spectral reflectance of the wavelengths between 640 nm and 650 nm makes it possible to increase the speed of determining a depth of invasion.

Note that the predetermined wavelength band may be set such that the wavelength of 645 nm is a median and, for example, the predetermined wavelength band may be set between the wavelength of 630 nm and the wavelength of 660 nm.

### EMBODIMENT 2-1

A process that the image processing apparatus executes according to Embodiment 2-1 will be described next. Description of the same process as the process already described will be omitted as appropriate.

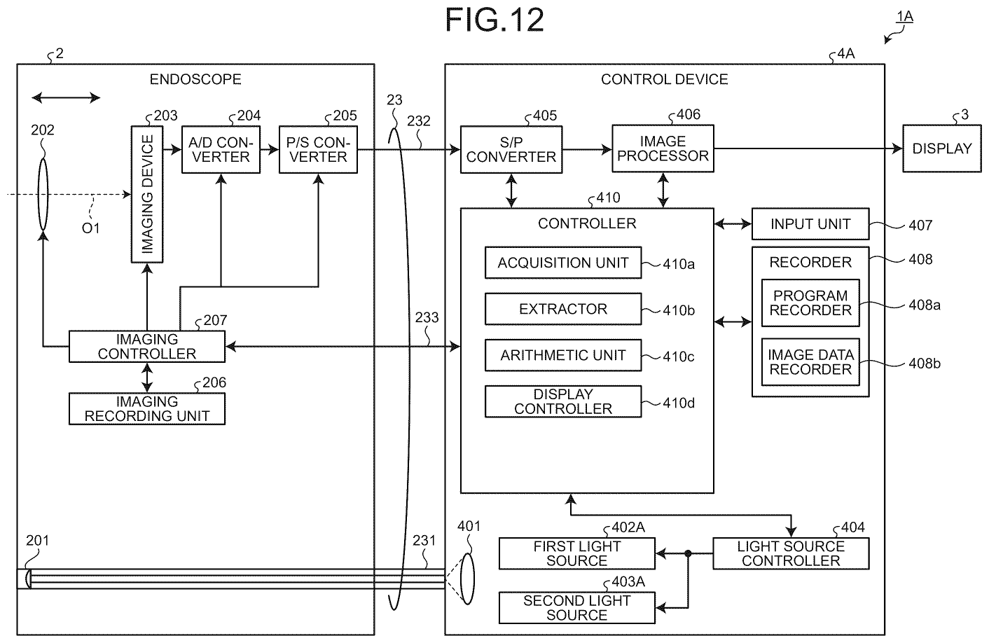

FIG. 12 is a block diagram illustrating a functional configuration of a relevant part of the endoscope and a control device according to Embodiment 2-1. As illustrated in FIG. 12, in an endoscope system 1A, a control device 4A includes a first light source 402A and a second light source 403A.

The first light source 402A may have the same configuration as that of the light source 402 and thus description thereof will be omitted.

Under the control of the light source controller 404, the second light source 403A emits special light having a predetermined wavelength band, thereby supplying special light as illuminating light to the light guide 231. The special light herein is, for example, red light (first observation light) having a peak wavelength near 630 nm and amber light (second observation light) having a peak wavelength near 600 nm that are used for red light observation (red dichromatic imaging (RDI)). The special light may be light used for narrowband observation (narrowband imaging (NBI)) using narrowband light containing the wavelengths from 390 nm to 445 nm and the wavelengths from 530 nm to 550 nm.

The acquisition unit 410a acquires a first observation image obtained by capturing an image of return light of the first observation light applied to a subject and a second observation image obtained by capturing an image of return light of the second observation light applied to the subject.

The arithmetic unit 410c estimates a depth of invasion based on the pixel value in each pixel of the first observation image and the pixel value in each pixel of the second observation image.

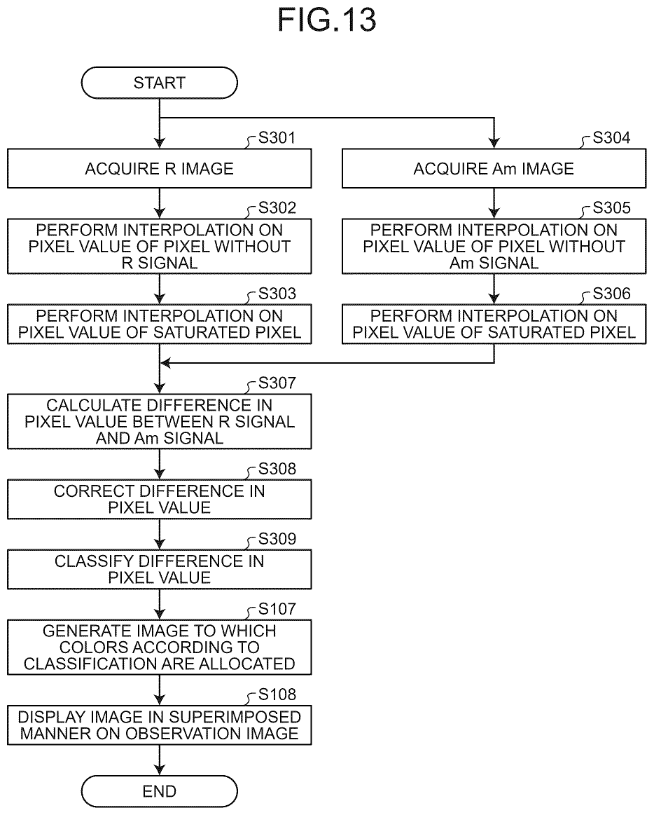

FIG. 13 is a flowchart illustrating an overview of a process that the image processing apparatus executes according to Embodiment 2-1.

As illustrated in FIG. 13, first of all, the acquisition unit 410a acquires an R image (first observation image) (step S301).

Subsequently, the arithmetic unit 410c performs interpolation on a pixel value of a pixel without an R signal (step S302).

Thereafter, as at step S102, the arithmetic unit 410c performs interpolation on a pixel value of a saturated pixel (step S303).

In parallel with steps S301 to S303, the process of steps S304 to S306 is executed. First of all, the acquisition unit 410a acquires an Am image (second observation image) (step S304).

The process of steps S305 and S306 is the same as the process of steps S302 and S303 and thus description thereof will be omitted.

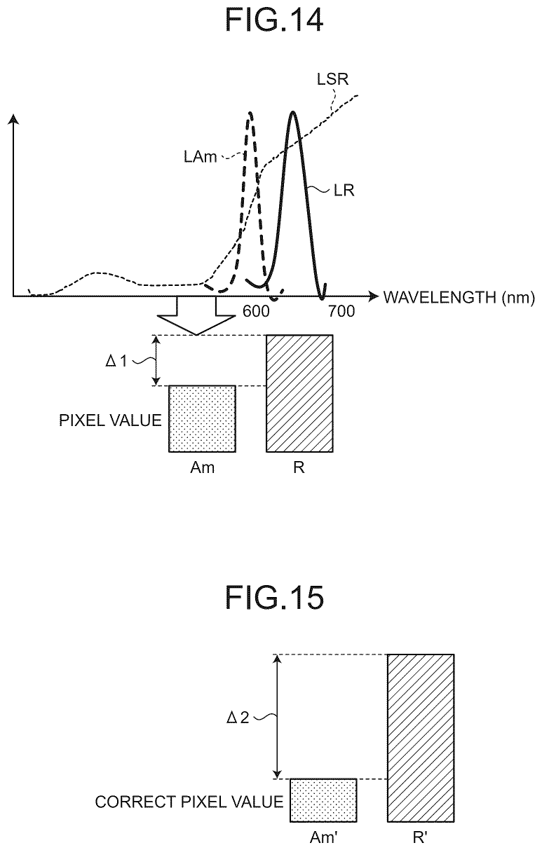

The arithmetic unit 410c calculates a difference in pixel value between an R signal and an Am signal (step S307). FIG. 14 is a diagram illustrating the difference in pixel value between the R signal and the Am signal. FIG. 14 illustrates a line LR representing red light and a line Lam representing amber light with respect to a line LSR representing the spectral reflectance. A difference Δ1 obtained by subtracting a pixel value Am of amber light from a pixel value R of red light corresponds to the slope of the spectroscopic spectrum and thus it is possible to estimate a depth of invasion using the magnitude of the difference Δ1.

Back to FIG. 13, the arithmetic unit 410c corrects the difference in pixel value (step S308). FIG. 15 is a diagram illustrating that the pixel values of the R signal and the Am signal is corrected. As illustrated in FIG. 15, the pixel value R of red light is corrected to a pixel value R′ and corrects the pixel value Am of amber light to a pixel value Am′ such that the difference Δ1<a difference Δ2. This post-processing corrects the difference Δ1 serving as an index of estimation of a depth of invasion. A method of calculation for correction is not particularly limited and, for example, raising each of the pixel values R and Am to a third power makes it possible to correct the pixel values such that the difference Δ1<the difference Δ2. Note that the pixel values R and Am may be raised to the second power or the fourth power or more.

Back to FIG. 13, the arithmetic unit 410c classifies the difference Δ2 in pixel value (step S309). The arithmetic unit 410c, for example, classifies the difference Δ2 in pixel value into SM1 or shallower or SM 2 or deeper.

Thereafter, as in Embodiment 1-1, the process of steps S107 and S108 is executed.

According to Embodiment 2-1 described above, it is possible to estimate a depth of invasion using only the two components of red light and amber light and thus it is usable generally in endoscopes equipped with an RDI function and makes it possible to reduce the load of processing.

Note that, in Embodiment 2-1, the example where the arithmetic unit 410c calculates the difference in pixel value between the R signal and the Am signal has been described, and the arithmetic unit 410c may calculate a ratio of the pixel values of the R signal and the Am signal and estimate a depth of invasion according to the calculated ratio.

### EMBODIMENT 2-2

A process that the image processing apparatus executes according to Embodiment 2-2 will be described next. Description of the same process as the process already described will be omitted as appropriate.

The arithmetic unit 410c estimate a depth of invasion based on the pixel value in each pixel of a first observation image.

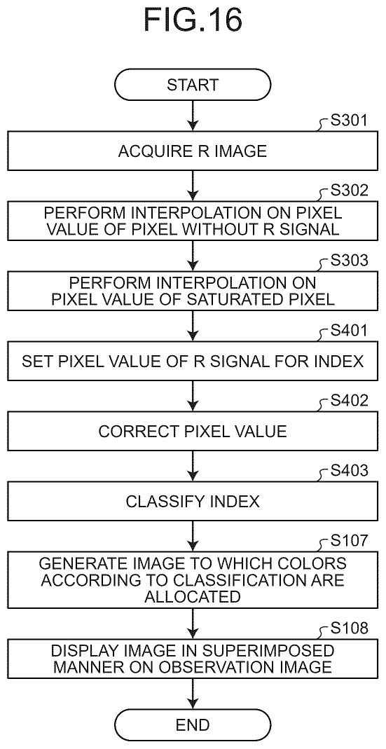

FIG. 16 is a flowchart illustrating an overview of a process that the image processing apparatus executes according to Embodiment 2-2. Steps S301 to S303 are the same as in Embodiment 2-1 and thus description thereof will be omitted.

Thereafter, the arithmetic unit 410c sets the pixel values of the R signal as an index (step S401). In Embodiment 2-1, the difference between the pixel value of red light and the pixel value of amber light is used as the index of a depth of invasion, and the pixel value of amber light is sufficiently smaller than the pixel value of red light. For this reason, the difference between the pixel value of amber light and the pixel value of red light may be approximated and the pixel value of the R signal may be set for the index for a depth of invasion.

Subsequently, the arithmetic unit 410c corrects the pixel value (step S402). The post-processing corrects the pixel value of the R signal serving as the index of estimation of a depth of invasion.

Furthermore, the arithmetic unit 410c classifies the index (S403). The arithmetic unit 410c, for example, classifies the pixel value of the R signal into SM1 or shallower or SM 2 or deeper.

Thereafter, as in Embodiment 1-1, the process of steps S107 and S108 is executed.

According to Embodiment 2-2 described above, it is possible to estimate a depth of invasion using only red light and thus it is possible to reduce the load of processing.

### EMBODIMENT 2-3

A process that the image processing apparatus executes according to Embodiment 2-3 will be described next. Description of the same process as the process already described will be omitted as appropriate.

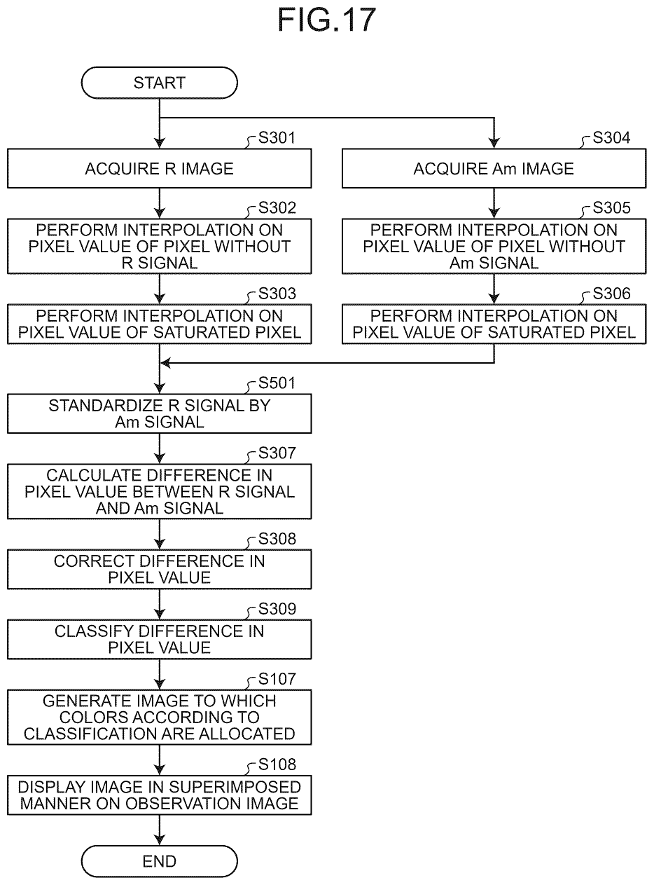

FIG. 17 is a flowchart illustrating an overview of the process executed by the image processing apparatus according to Embodiment 2-3. Steps S301 to S306 are the same as in Embodiment 2-1 and thus description thereof will be omitted.

Thereafter, as illustrated in FIG. 17, the arithmetic unit 410c standardizes the pixel value in each pixel of the R image (first observation image) by the pixel value in each pixel of the Am image (second observation image) (step S501). Standardizing the pixel value of the R signal by the pixel value of the Am signal in each pixel and thereafter calculating the difference make it possible to present the slope of the spectroscopic spectrum remarkably.

The subsequent process is the same as in Embodiment 2-1 and thus description thereof will be omitted.

According to Embodiment 2-3 described above, standardizing the pixel value of the R signal by the pixel value of the Am signal makes it possible to increase the speed of determining a depth of invasion.

### EMBODIMENT 2-4

A process that the image processing apparatus executes according to Embodiment 2-4 will be described next. Description of the same process as the process already described will be omitted as appropriate.

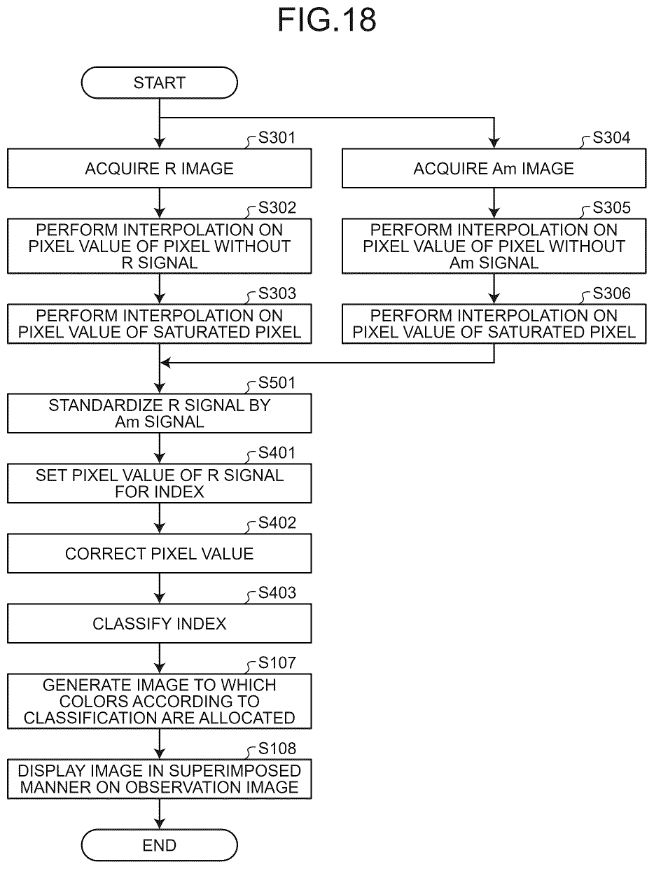

FIG. 18 is a flowchart illustrating an overview of the process executed by the image processing apparatus according to Embodiment 2-4. Steps S301 to S306 are the same as in Embodiment 2-1 and thus description thereof will be omitted.

Thereafter, as illustrated in FIG. 18, the arithmetic unit 410c standardizes the pixel value of the R signal by the pixel value of the Am signal (step S501). As in Embodiment 2-3, standardizing the pixel value of the R signal by the pixel value of the Am signal in each pixel makes it possible to present the slope of the spectroscopic spectrum remarkably.

The subsequent process is the same as in Embodiment 2-2 and thus description thereof will be omitted.

According to Embodiment 2-4 described above, standardizing the pixel value of the R signal by the pixel value of the Am signal makes it possible to increase the speed of determining a depth of invasion.

### EMBODIMENT 3-1

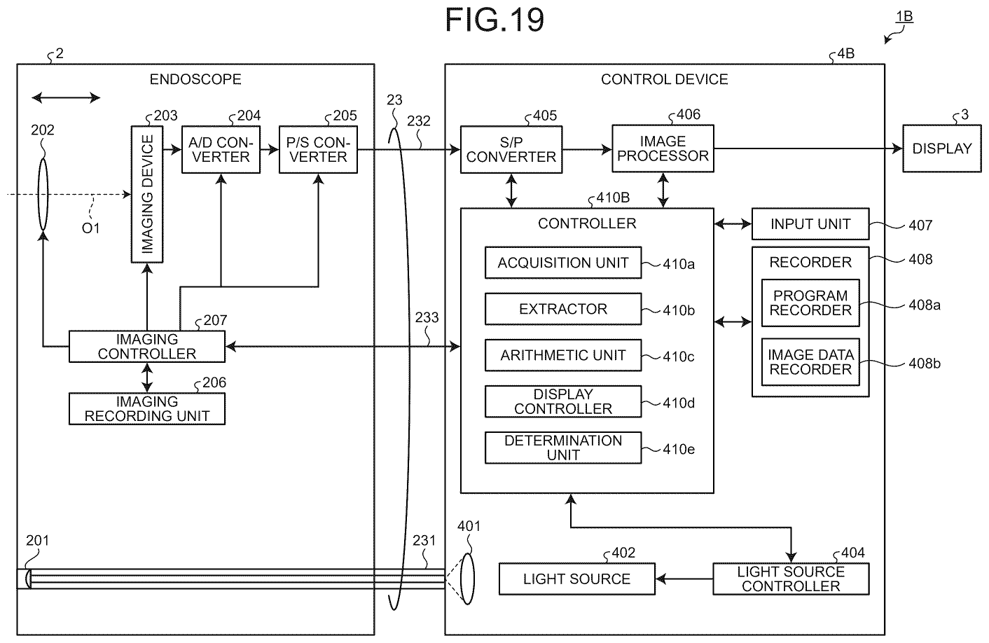

A process that the image processing apparatus executes according to Embodiment 3-1 will be described next. Description of the same process as the process already described will be omitted as appropriate. FIG. 19 is a block diagram illustrating a functional configuration of a relevant part of an endoscope and a control device according to Embodiment 3-1. As illustrated in FIG. 19, in an endoscope system 1B, a controller 410B of a control device 4B includes a determination unit 410e.

Based on the depth of invasion, the determination unit 410e determines whether it is an area that is highly likely to be cancerous.

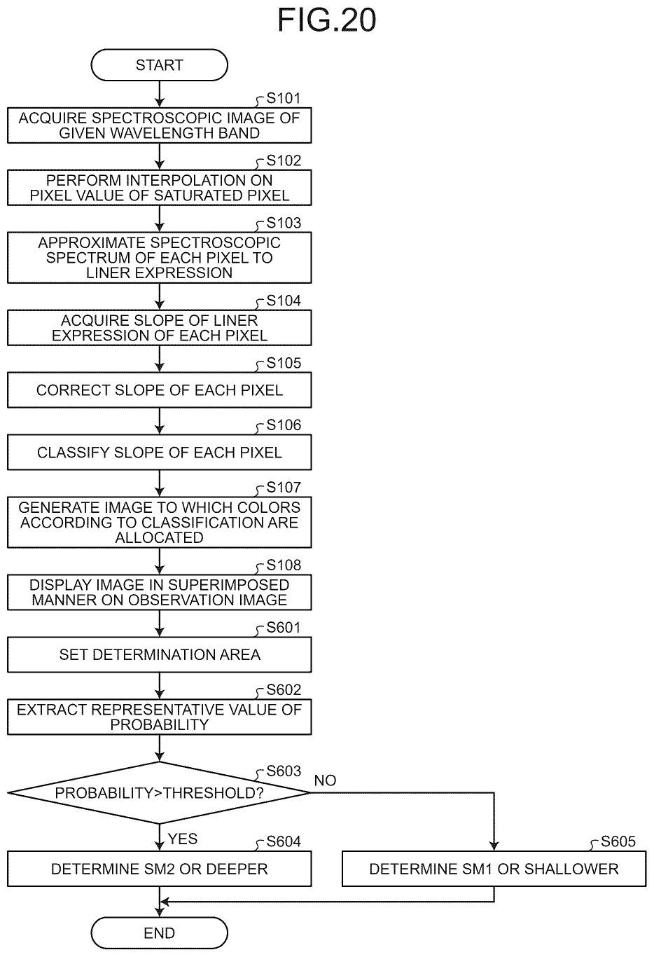

FIG. 20 is a flowchart illustrating an overview of a process that an image processing apparatus executes according to Embodiment 3-1. Steps S101 to S108 are the same as in Embodiment 1-1 and thus description thereof will be omitted.

Thereafter, the arithmetic unit 410c sets a determination area on which it is determined whether the area is SM2 or deeper or SM1 or shallower according to an input made by the user to the input unit 407 (step S601). Note that an area that is set automatically according to a predetermined program may be set for the determination area. A plurality of possible areas may be extracted according to a predetermined program and an area that is selected by the user from the possible areas may be set for the determination area.

Subsequently, the arithmetic unit 410c extracts a representative value of probability of the determination area (step S602). The probability is probability that the depth of invasion that is calculated based on the slope of that is calculated at step S106 is SM2 or deeper. The representative value may be an average of probabilities of each pixel of the determination area and the representative value may be a median, a mode, or a maximum of probabilities of each pixel of the determination area. The representative value may be a value that is set by the user or may be a value that is selected by the user from a plurality of possible representative values that are calculated automatically according to a predetermined program.

The determination unit 410e then determines whether the probability>a threshold (step S603).

When the determination unit 410e determines that the probability>the threshold (Yes at step S603), the determination unit 410e determines that the depth of invasion in the determination area is SM2 or deeper and outputs the result of the determination (step S604).

When the determination unit 410e does not determine that the probability>the threshold (No at step S603), the determination unit 410e determines that the depth of invasion in the determination area is SM1 or shallower and outputs the result of the determination (step S605).

According to Embodiment 3-1 described above, the determination unit 410e outputs the determination result and thus the endoscopist refers to the determination result, which makes it possible to shorten the time required to determine a depth of invasion.

### EMBODIMENT 3-2

A process that the image processing apparatus executes according to Embodiment 3-2 will be described next. Description of the same process as the process already described will be omitted as appropriate.

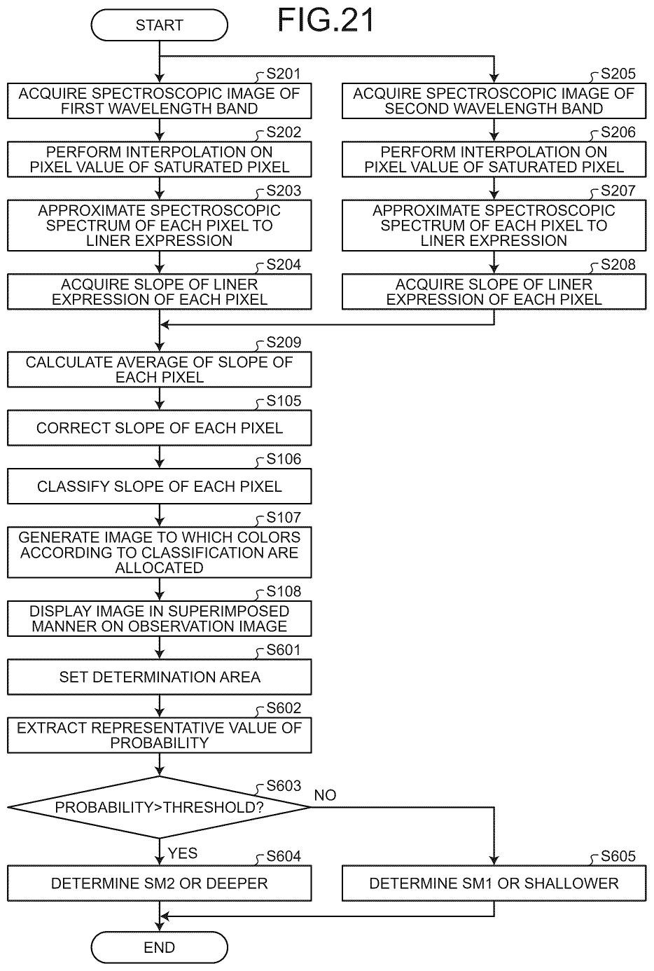

FIG. 21 is a flowchart illustrating an overview of a process that an image processing apparatus executes according to Embodiment 3-2. Steps S201 to S108 are the same as in Embodiment 2-1 and thus description thereof will be omitted.

Thereafter, as in Embodiment 3-1, the process of steps S601 to S605 is executed.

According to Embodiment 3-2 described above, the determination unit 410e outputs the determination result and thus the endoscopist refers to the determination result, which makes it possible to shorten the time required to determine a depth of invasion.

### EMBODIMENT 3-3

A process that the image processing apparatus executes according to Embodiment 3-3 will be described next. Description of the same process as the process already described will be omitted as appropriate. The process that the image processing apparatus executes according to Embodiment 3-3 may be the same as the process of Embodiment 3-1 illustrated in FIG. 20 and thus description thereof will be omitted. Note that, at step S101, the acquisition unit 410a acquires a spectroscopic image of a predetermined wavelength band (from the wavelength of 640 nm to the wavelength of 650 nm) that is extracted by the extractor 410b (step S101).

According to Embodiment 3-3 described above, as in Embodiment 1-3, calculating a spectroscopic slope at the wavelength of 645 nm makes it possible to increase the speed of determining a depth of invasion and, as in Embodiment 3-1, the determination unit 410e outputs the determination result and thus the endoscopist refers to the determination result, which makes it possible to shorten the time required to determine a depth of invasion.

### EMBODIMENT 4-1

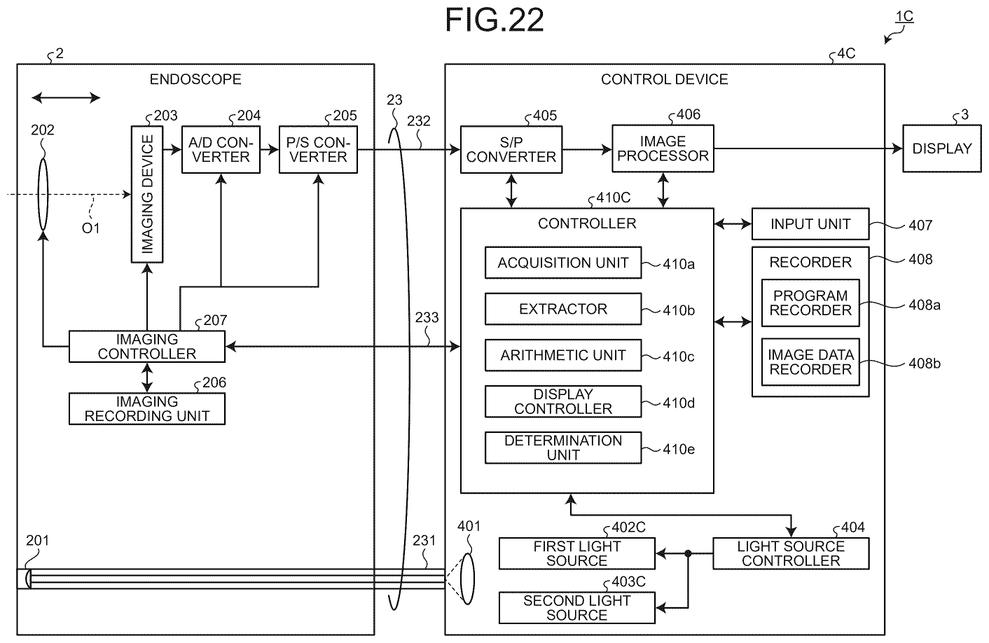

A process that the image processing apparatus executes according to Embodiment 4-1 will be described next. Description of the same process as the process already described will be omitted as appropriate. FIG. 22 is a block diagram illustrating a functional configuration of a relevant part of the endoscope and a control device according to Embodiment 4-1. As illustrated in FIG. 22, in an endoscope system 1C, a control device 4C includes a first light source 402C and a second light source 403C. A controller 410C of the control device 4C includes a determination unit 410e.

The first light source 402C and the second light source 403C may have the same configurations as those of the first light source 402A and thus description thereof will be omitted.

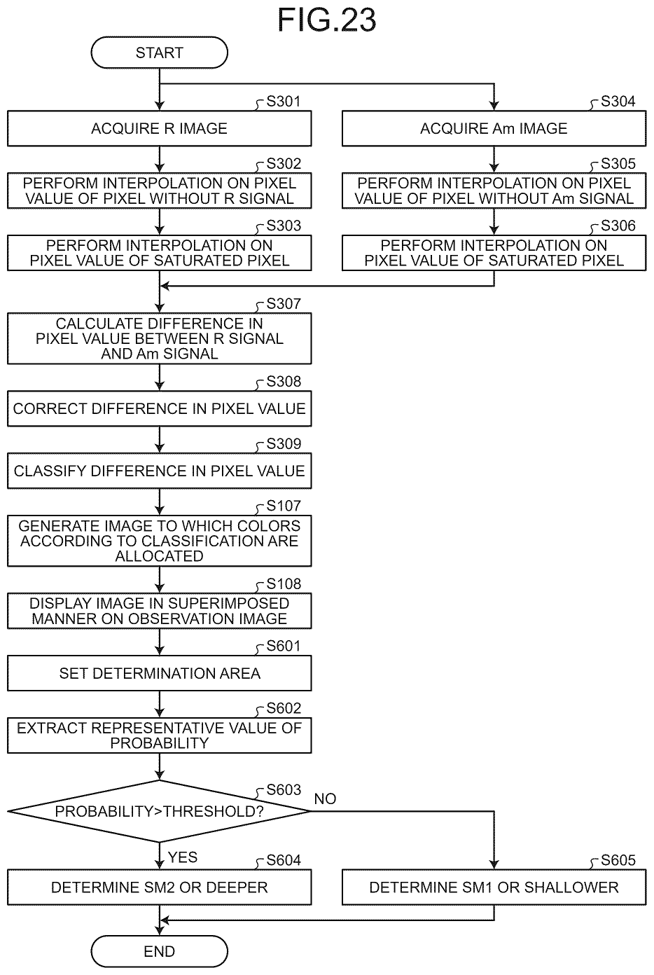

FIG. 23 is a flowchart illustrating an overview of a process that the image processing apparatus executes according to Embodiment 4-1. Steps S301 to S108 are the same as in Embodiment 2-1 and thus description thereof will be omitted.

Thereafter, the process of steps S601 to S605 is executed as in Embodiment 3-1.

According to Embodiment 4-1 described above, the determination unit 410e outputs the determination result and thus the endoscopist refers to the determination result, which makes it possible to shorten the time required to determine a depth of invasion.

### EMBODIMENT 4-2

A process that the image processing apparatus executes according to Embodiment 4-2 will be described next. Description of the same process as the process already described will be omitted as appropriate.

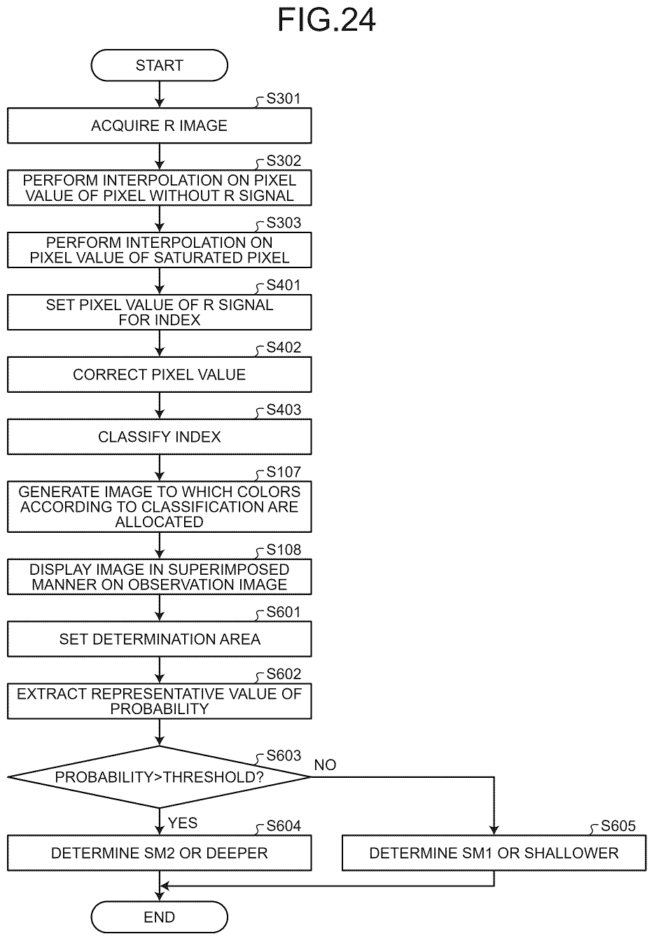

FIG. 24 is a flowchart illustrating an overview of a process that the image processing apparatus executes according to Embodiment 4-2. As illustrated in FIG. 24, steps S301 to S108 are the same as in Embodiment 2-2 and thus description thereof will be omitted.

Thereafter, as in Embodiment 3-1, the process of steps S601 to S605 is executed.

According to Embodiment 4-2 described above, the determination unit 410e outputs the determination result and thus the endoscopist refers to the determination result, which makes it possible to shorten the time required to determine a depth of invasion.

### EMBODIMENT 4-3

A process that the image processing apparatus executes according to Embodiment 4-3 will be described next. Description of the same process as the process already described will be omitted as appropriate.

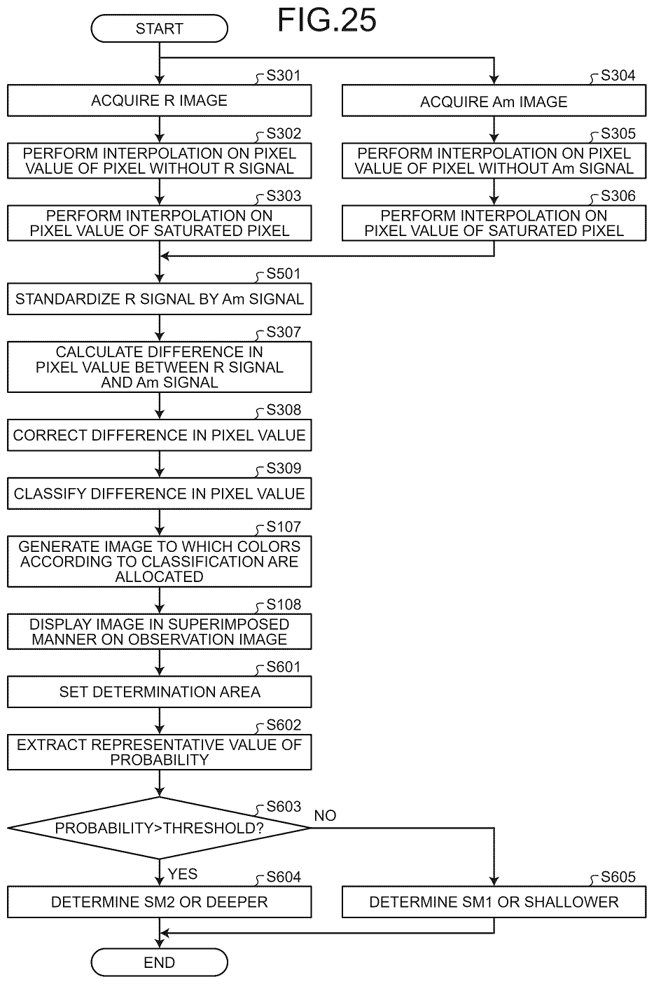

FIG. 25 is a flowchart illustrating an overview of a process that the image processing apparatus executes according to Embodiment 4-3. Steps S301 to S108 are the same as in Embodiment 2-3 and thus description thereof will be omitted.

Thereafter, as in Embodiment 3-1, the process of steps S601 to S605 is executed.

According to Embodiment 4-3 described above, the determination unit 410e outputs the determination result and thus the endoscopist refers to the determination result, which makes it possible to shorten the time required to determine a depth of invasion.

### EMBODIMENT 4-4

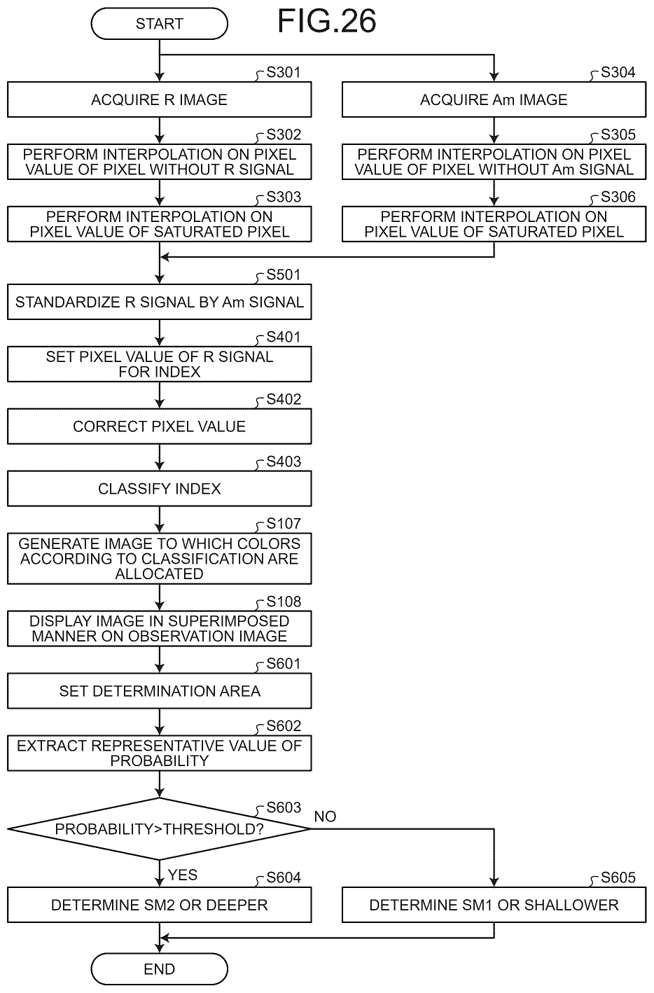

A process that the image processing apparatus executes according to Embodiment 4-4 will be described next. Description of the same process as the process already described will be omitted as appropriate. FIG. 26 is a flowchart illustrating an overview of a process that the image processing apparatus executes according to Embodiment 4-4. As illustrated in FIG. 26, steps S301 to S108 are the same as in Embodiment 2-4 and thus description thereof will be omitted.

Thereafter, as in Embodiment 3-1, the process of steps S601 to S605 is executed.

According to Embodiment 4-4 described above, the determination unit 410e outputs the determination result and thus the endoscopist refers to the determination result, which makes it possible to shorten the time required to determine a depth of invasion.

### EMBODIMENT 5-1

A process that the image processing apparatus executes according to Embodiment 5-1 will be described next. Description of the same process as the process already described will be omitted as appropriate.

The acquisition unit 410a acquires a normal light observation image obtained by capturing an image of return light of white light applied to a subject.

The arithmetic unit 410c estimates a depth of invasion based on the pixel value of each pixel of a first observation image and the pixel value of each pixel of the normal image.

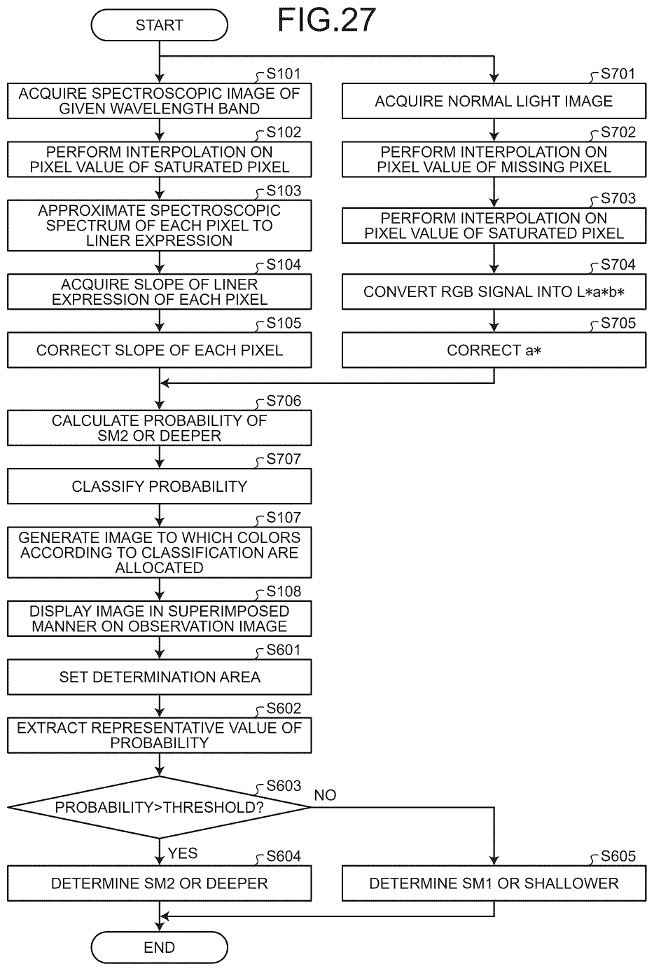

FIG. 27 is a flowchart illustrating an overview of a process that the image processing apparatus executes according to Embodiment 5-1. As illustrated in FIG. 27, in parallel with steps S101 to S105, the process of steps S701 to S705 is executed. First of all, the acquisition unit 410a acquires a normal light image (step S701).

Subsequently, the arithmetic unit 410c performs interpolation on a pixel value of a missing pixel of the normal light image (step S702).

Thereafter, as at step S102, the arithmetic unit 410c performs interpolation on a pixel value of a saturated pixel (step S703). The pre-processing of steps S702 and S703 reduces variations among the pixels.

The arithmetic unit 410c then performs color conversion into L*a*b* on an RGB signal (step S704).

Furthermore, the arithmetic unit 410c then corrects a* corresponding to red in a L*a*b* color space (step S705).

The arithmetic unit 410c calculates a probability of SM2 or deeper (step S706). First of all, using two expressions presented by (Equation 1) y=α×(a slope)+β×(a*)+γ and (Equation 2) P=e^{y}/(1+e^{y})=1/(1+e−^{y}) and few tens to few hundreds of sets of data on which doctors, and the like, have determined depths of cancerous invasion (for example, data on which SM2 or deeper (P=1) and SM1 or shallower (P=0) are known), the arithmetic unit 410c calculates coefficients α, β and γ in (Equation 1) by logistic regression analysis. Note that, for (the slope) and (a*) in (Equation 1), values obtained by performing the same corrections as those at steps S105 and S705 are used. By assigning a corrected value of the slope that is calculated at step S105 and a corrected value of a* that is calculated at step S705 to the equation, it is possible to calculate a probability P.

Thereafter, the arithmetic unit 410c classifies the probability (step S707). The arithmetic unit 410c classifies the probability into being at or above a threshold or being under the threshold.

Thereafter, as in Embodiment 3-1, the process of steps S107 to S605 is executed.

According to Embodiment 5-1 described above, a depth of invasion is determined using a* in addition to Embodiment 3-1 and thus it is possible to increase the speed of determining a depth of invasion.

### EMBODIMENT 5-2

A process that the image processing apparatus executes according to Embodiment 5-2 will be described next. Description of the same process as the process already described will be omitted as appropriate.

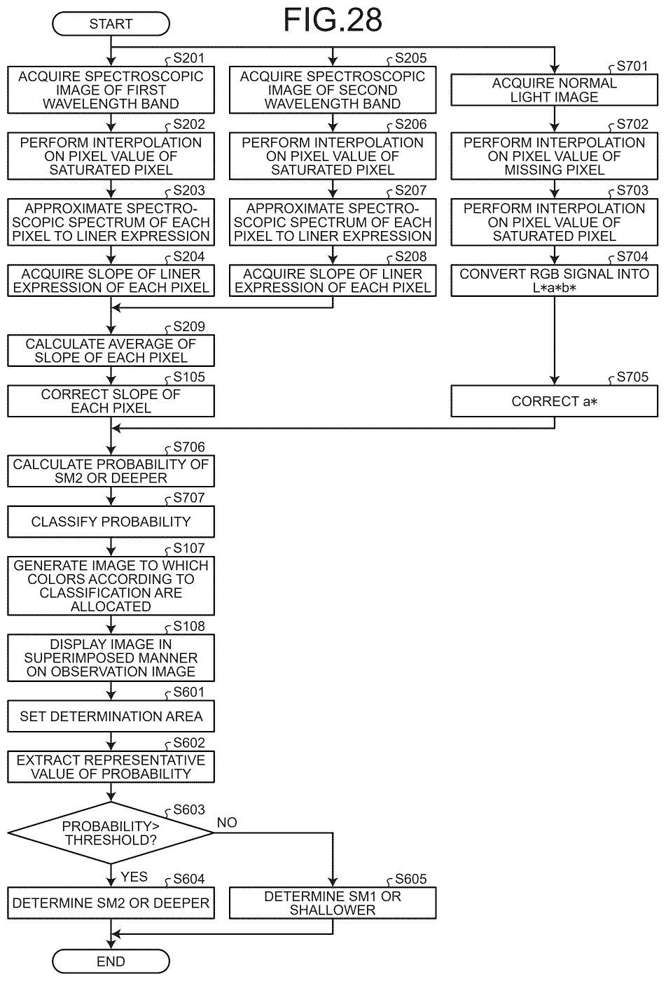

FIG. 28 is a flowchart illustrating an overview of a process that the image processing apparatus executes according to Embodiment 5-2. In Embodiment 5-2, the same processes as the process in Embodiment 3-2 illustrated in FIG. 21 and the process in Embodiment 5-1 illustrated in FIG. 27 are executed.

According to Embodiment 5-1 described above, a depth of invasion is determined using a* in addition to Embodiment 3-2 and thus it is possible to increase the speed of determining a depth of invasion.

EMBODIMENT 5-3

A process that the image processing apparatus executes according to Embodiment 5-3 will be described next. Description of the same process as the process already described will be omitted as appropriate. The process executed by the image processing apparatus according to Embodiment 5-3 may be the same as the process of Embodiment 5-1 illustrated in FIG. 27 and thus description thereof will be omitted. Note that, at step S101, the acquisition unit 410a acquires a spectroscopic image of a predetermined wavelength band (from the wavelength of 640 nm to the wavelength of 650 nm) that is extracted by the extractor 410b (step S101).

According to Embodiment 5-3 described above, a depth of invasion is determined using a* in addition to Embodiment 3-3 and thus it is possible to increase the speed of determining a depth of invasion.

### EMBODIMENT 6-1

A process that the image processing apparatus executes according to Embodiment 6-1 will be described next. Description of the same process as the process already described will be omitted as appropriate.

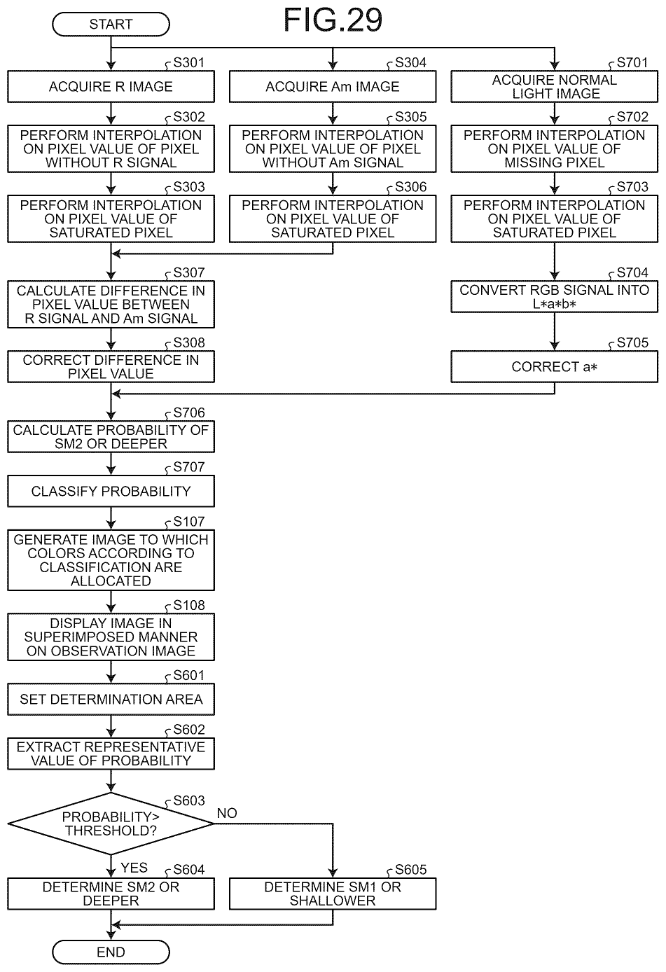

FIG. 29 is a flowchart illustrating an overview of a process that the image processing apparatus executes according to Embodiment 6-1. As illustrated in FIG. 29, in Embodiment 6-1, the same processes as the process in Embodiment 4-1 illustrated in FIG. 23 and the process in Embodiment 5-1 illustrated in FIG. 27 are executed.

According to Embodiment 6-1 described above, a depth of invasion is determined using a* in addition to Embodiment 4-1 and thus it is possible to increase the speed of determining a depth of invasion.

### EMBODIMENT 6-2

A process that the image processing apparatus executes according to Embodiment 6-2 will be described next. Description of the same process as the process already described will be omitted as appropriate.

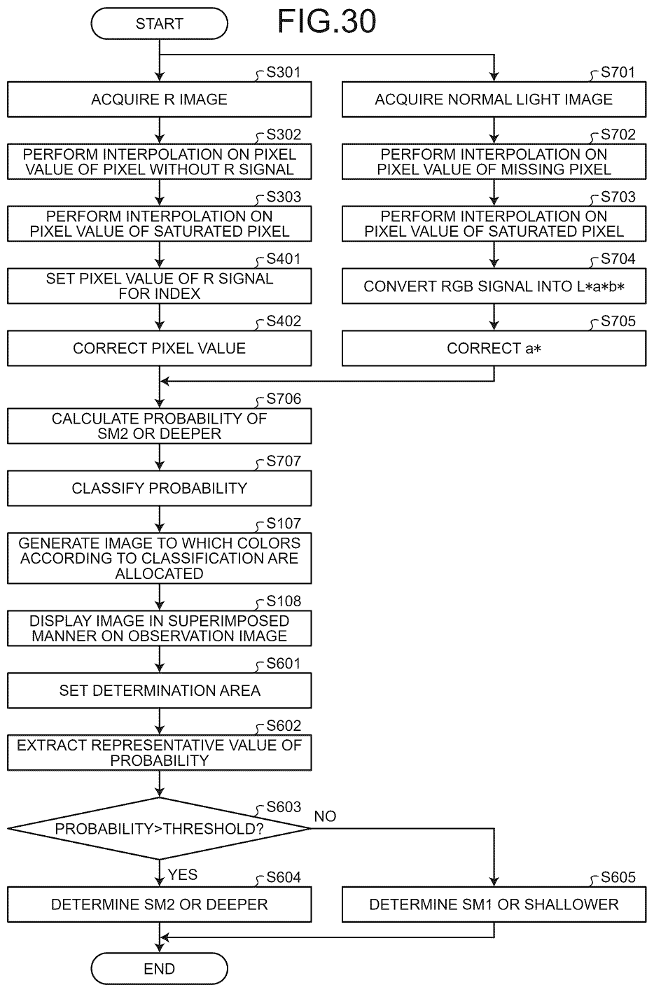

FIG. 30 is a flowchart illustrating an overview of a process that the image processing apparatus executes according to Embodiment 6-2. As illustrated in FIG. 30, in Embodiment 6-2, the same processes as the process in Embodiment 4-2 illustrated in FIG. 24 and the process in Embodiment 5-1 illustrated in FIG. 27 are executed.

According to Embodiment 6-2 described above, a depth of invasion is determined using a* in addition to Embodiment 4-2 and thus it is possible to increase the speed of determining a depth of invasion.

### EMBODIMENT 6-3

A process that the image processing apparatus executes according to Embodiment 6-3 will be described next. Description of the same process as the process already described will be omitted as appropriate.

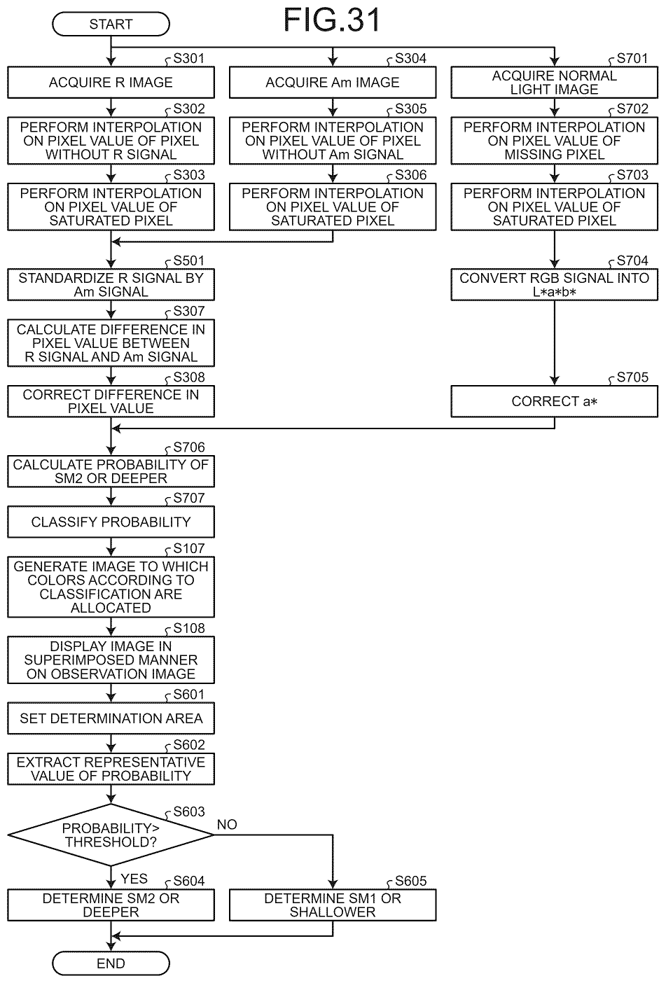

FIG. 31 is a flowchart illustrating an overview of a process that the image processing apparatus executes according to Embodiment 6-3. As illustrated in FIG. 31, in Embodiment 6-3, the same processes as the process in Embodiment 4-3 illustrated in FIG. 25 and the process in Embodiment 5-1 illustrated in FIG. 27 are executed.

According to Embodiment 6-3 described above, a depth of invasion is determined using a* in addition to Embodiment 4-3 and thus it is possible to increase the speed of determining a depth of invasion.

### EMBODIMENT 6-4

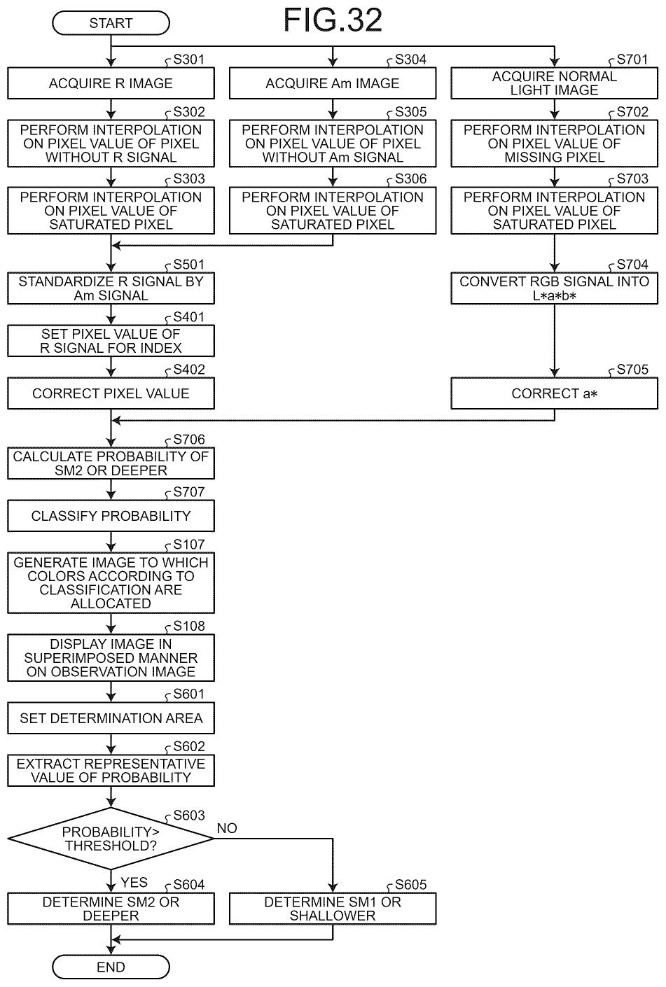

A process that the image processing apparatus executes according to Embodiment 6-4 will be described next. Description of the same process as the process already described will be omitted as appropriate. FIG. 32 is a flowchart illustrating an overview of a process that the image processing apparatus executes according to Embodiment 6-4. As illustrated in FIG. 32, in Embodiment 6-4, the same processes as the process in Embodiment 4-4 illustrated in FIG. 26 and the process in Embodiment 5-1 illustrated in FIG. 27 are executed.

According to Embodiment 6-4 described above, a depth of invasion is determined using a* in addition to Embodiment 4-4 and thus it is possible to increase the speed of determining a depth of invasion.

According to the disclosure, it is possible to realize an image processing apparatus, an endoscope system, and an operation method performed by an image processing apparatus that make it possible to increase a speed of determining a depth of invasion.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing apparatus comprising: a processor comprising hardware, the processor being configured to
acquire a first observation image obtained by capturing an image of return light of first observation light applied to a subject; and
estimate a depth of invasion based on a pixel value of each pixel of the first observation image.

2. The image processing apparatus according to claim 1, wherein the first observation light is wideband light having a wideband wavelength component, and
the first observation image is a spectroscopic image of each wavelength.

3. The image processing apparatus according to claim 2, wherein, the processor is further configured to
extract the pixel value of each pixel of the spectroscopic image in a predetermined wavelength band, and
estimate the depth of invasion based on a slope of a spectroscopic spectrum that is generated from the extracted pixel value.

4. The image processing apparatus according to claim 3, wherein the predetermined wavelength band is contained in wavelengths from 600 nm to 700 nm.

5. The image processing apparatus according to claim 3, wherein a lower limit of the predetermined wavelength band is a wavelength where a difference in change between an absorption coefficient of deoxygenated hemoglobin and an absorption coefficient of oxygenated hemoglobin at each wavelength occurs.

6. The image processing apparatus according to claim 2, wherein, the processor is further configured to
extract the pixel value of each pixel of the spectroscopic image in each of two wavelength bands, and
estimate the depth of invasion based on the extracted pixel value.

7. The image processing apparatus according to claim 6, wherein the two wavelength bands are contained in wavelengths from 600 nm to 700 nm.

8. The image processing apparatus according to claim 6, wherein lower limits of the two wavelength bands are a wavelength where a difference in change between an absorption coefficient of deoxygenated hemoglobin and an absorption coefficient of oxygenated hemoglobin at each wavelength occurs.

9. The image processing apparatus according to claim 1, wherein the processor is further configured to acquire a second observation image obtained by capturing an image of return light of second observation light applied to the subject,
the first observation light has a peak wavelength near a wavelength of 630 nm, and
the second observation light has a peak wavelength near a wavelength of 600 nm.

10. The image processing apparatus according to claim 9, wherein the processor is configured to estimate the depth of invasion based on the pixel value of each pixel of the first observation image and a pixel value of each pixel of the second observation image.

11. The image processing apparatus according to claim 9, wherein the processor is further configured to standardize the pixel value of each pixel of the first observation image by a pixel value of each pixel of the second observation image.

12. The image processing apparatus according to claim 9, wherein the processor is configured to estimate the depth of invasion based on the pixel value of each pixel of the first observation image.

13. The image processing apparatus according to claim 1, wherein the processor is further configured to
acquire a normal light image obtained by capturing an image of return light of white observation light applied to the subject, and
estimate the depth of invasion based on the pixel value of each pixel of the first observation image and a pixel value of each pixel of the normal light image.

14. The image processing apparatus according to claim 1, wherein the processor is further configured to cause a display to display the depth of invasion of each pixel.

15. The image processing apparatus according to claim 14, wherein the processor is further configured to cause the display to display the depth of invasion of each pixel in a superimposed manner on the first observation image or a normal light image.

16. The image processing apparatus according to claim 1, wherein the processor is further configured to determine whether it is an area that is highly likely to be cancerous based on the depth of invasion of each pixel.

17. An endoscope system comprising:
a light source configured to apply first observation light to a subject from a distal end of an insertion portion to be inserted into the subject;
an imager configured to generate a first observation image obtained by capturing an image of return light of the first observation light; and
an image processing apparatus comprising a processor comprising hardware, the processor being configured to acquire the first observation image from the imager and estimate a depth of invasion based on a pixel value of each pixel of the first observation image.

18. The endoscope system according to claim 17, wherein the first observation light is wideband light having a wideband wavelength component,
the first observation image is a spectroscopic image of each wavelength,
the processor of the image processing apparatus is further configured to extract the pixel value of each pixel of the spectroscopic image in a predetermined wavelength band and estimate the depth of invasion based on a slope of a spectroscopic spectrum that is generated from the extracted pixel value.

19. The endoscope system according to claim 17, wherein
the light source is further configured to apply second observation light to the subject from the distal end of the insertion portion,
the imager is further configured to generate a second observation image by capturing an image of return light of the second observation light,
the processor of the image processing apparatus is configured to
acquire the first observation image and the second observation image from the imager, and
estimate the depth of invasion based on the pixel value of each pixel of the first observation image and a pixel value of each pixel of the second observation image.

20. An operation method performed by an image processing apparatus, the method comprising:
acquiring a first image obtained by capturing an image of return light of first observation light applied to a subject; and
estimating a depth of invasion based on a pixel value of each pixel of the first observation image.

21. The operation method according to claim 20, wherein
the first observation light is wideband light having a wideband wavelength component,
the first observation image is a spectroscopic image of each wavelength,
the method further comprises extracting the pixel value of each pixel of the spectroscopic image in a predetermined wavelength band, and
the estimating includes estimating the depth of invasion based on a slope of a spectroscopic spectrum that is generated from the pixel value extracted by the extracting.

22. The operation method according to claim 20, wherein the acquiring includes acquiring a second observation image obtained by capturing an image of return light of second observation light applied to the subject, and
the estimating includes estimating the depth of invasion based on the pixel value of each pixel of the first observation image and a pixel value of each pixel of the second observation image.
